(19) United States
(12) Patent Application Publication
(10) Pub. No.: US 2013/0158371 A1
(43) Pub. Date: Jun. 20, 2013

(54) BIOELECTRICAL IMPEDANCE MEASURING APPARATUS, SEMICONDUCTOR DEVICE, AND CONTROL METHOD FOR BIOELECTRICAL IMPEDANCE MEASURING APPARATUS

(71) Applicant: Renesas Electronics Corporation, Kawasaki-shi (JP)

(72) Inventors: Mitsuaki MINETA, Yokohama-Shi (JP); Makoto ASAUMI, Yokohama-Shi (JP)

(73) Assignee: RENESAS ELECTRONICS CORPORATION, Kawasaki-shi (JP)

(21) Appl. No.: 13/722,823

(22) Filed: Dec. 20, 2012

(30) Foreign Application Priority Data
Dec. 20, 2011 (JP) 2011-277879

(51) Int. Cl. A61B 5/053

(52) U.S. Cl. CPC A61B 5/0537
USPC 600/305

(57) ABSTRACT

A current supply unit supplies a current between first and second current output terminals. A voltage measurement unit measures a voltage input to first and second voltage input terminals. A control circuit causes a first switch unit to connect the first current output terminal to a first terminal of a reference resistor and to connect the second current output terminal to a second terminal of the reference resistor, causes a second switch unit to connect the first voltage input terminal to the first terminal of the reference resistor and to connect the second voltage input terminal to the second terminal of the reference resistor, and causes a voltage measurement unit to measure a voltage between the first and second voltage input terminals, thereby performing self-diagnosis of a path through which a current flows.

### CROSS-REFERENCE TO RELATED APPLICATIONS

This application is based upon and claims the benefit of priority from Japanese patent application No. 2011-277879, filed on Dec. 20, 2011, the disclosure of which is incorporated herein in its entirety by reference.

### BACKGROUND

The present invention relates to a bioelectrical impedance measuring apparatus, a semiconductor device, and a control method for a bioelectrical impedance measuring apparatus. In particular, the present invention relates to a bioelectrical impedance measuring apparatus, a semiconductor device, and a control method for a bioelectrical impedance measuring apparatus, which are capable of preventing an overcurrent.

It is generally known that a body composition can be estimated by measuring the impedance of a living organism (Henry C Lukaski at. el., “Assessment of fat-free mass using bioelectrical impedance measurement of the human body”, The American Journal of Clinical Nutrition, 41, April 1985, pp. 810-817.). A technique for measuring a fat mass contained in a body by using this principle has been established. In this fat mass measurement technique, an impedance between terminals, such as limbs of a body, is measured by a four-terminal electrode method. Then, the fat mass is calculated based on personal body information such as the weight, height, gender, age, and the like of a person to be measured, and the measured impedance value. Apparatuses that measure the fat mass and the weight of a person to be measured at the same time by applying such a fat mass measurement technique are in widespread use (Japanese Unexamined Patent Application Publication No. 62-169023).

In the fat mass measurement, a current flowing into a living organism, such as a human body, may increase due to occurrence of a failure in a circuit element or expiration of the lifetime of a circuit element. However, when an excessive current exceeding a certain level flows through the living organism, a large load is applied to the living organism. For this reason, there is a demand for taking measures for preventing an overcurrent so as to prevent an adverse effect on the living organism. In fact, a technique for preventing an overcurrent from flowing during the fat mass measurement has already been proposed (Japanese Unexamined Patent Application Publication No. 2001-161655).

A body fat scale disclosed in Japanese Unexamined Patent Application Publication No. 2001-161655, which illustrates an example of a bioelectrical impedance measuring apparatus, will be described below. FIG. 7 is a block diagram illustrating an internal measurement circuit of a body fat scale 300 disclosed in Japanese Unexamined Patent Application Publication No. 2001-161655. The internal measurement circuit of the body fat scale includes a CPU 30 that performs various arithmetic processings and control processing. A sine-wave generation unit 31 that generates an alternating current to be measured is controlled by a processing instruction from the CPU 30. An output signal from the sine-wave generation unit 31 is supplied to a constant current circuit unit 32 including a constant current circuit operational amplifier U12 that generates a constant current. An output terminal of the constant current circuit unit 32 is connected to a current electrode 50B through a capacitor C1. Another terminal of the constant current circuit unit 32 is connected to a current electrode 50A through a reference resistor 33 and a capacitor C2.

Voltage electrodes 51A and 51B are connected to an analog switch IC1 through capacitors C3 and C4, respectively. This analog switch IC1 is connected to the reference resistor 33, and performs switching between measurement of the resistance of the reference resistor 33 and measurement of the impendence of a body. This reference resistor 33 is provided within a current path for a measurement current. A current is caused to flow through a certain path including both feet of a person to be measured. Then, a voltage value obtained when the reference resistor 33 is connected and a voltage value obtained upon switching to the path between both feet of the living organism are measured, thereby making it possible to compare the voltage with the reference resistor 33 which is accurately obtained. This allows a more accurate measurement of a bioelectrical impedance. The analog switch IC1 is connected to a voltage amplifying circuit unit 34 including a voltage amplifying operational amplifier U14 that amplifies a measured voltage value signal. The voltage amplifying circuit unit 34 is connected to a detector circuit 35 that shapes the voltage waveform. An A/D converter 36 converts data with a shaped voltage waveform from an analog value to a digital value. The digital value generated by the A/D converter 36 is input to the CPU 30.

The CPU 30 is connected with a switch group 37 including a numerical value change button, a set button, and a measurement start switch. The CPU 30 is also connected with a display circuit 38. The display circuit 38 includes a display unit that displays information on body fat, such as the percentage of body fat or a fat mass, which is estimated based on the obtained bioelectrical impedance value and the personal information, which is preliminarily set and stored, of a person to be measured. Accordingly, the CPU 30 calculates a bioelectrical impedance value from an input measurement current value and a detected measured voltage value, estimates the percentage of body fat or a fat mass based on the stored and set personal information and the obtained bioelectrical impedance value, and displays the estimation result on the display unit of the display circuit 38.

A battery 39 is connected to a constant voltage supply unit 40, and the entire measurement circuit is driven by a rated voltage (VDD) which is generated and output by the constant voltage supply unit 40. The battery 39 is connected to each of the elements including the CPU 30, and supplies a constant voltage to each of the elements. This constant voltage supply unit 40 is generally configured by combining a three-terminal regulator and the like, and outputs rated voltages VDD and −VDD.

Diodes D11 to D14 and D21 to D24 are provided for countermeasures against electrostatic breakdown. For example, when socks, clothing, and the like electrically charged by a carpet or the like contact the current electrodes 50A and 50B and the voltage electrodes 51A and 51B, a voltage of several kV may be applied from the electrodes due to static electricity. To prevent breakdown of the circuit due to such a voltage, the diodes D11 to D14 and D21 to 24 are provided to allow the current to flow to VDD or −VDD when a high voltage is applied between each electrode and the measurement circuit. This prevents an excessive current from flowing to the circuit side.

Here, a description is given of a case where a failure occurs in a circuit element in the circuit in which the capacitors C1 to C4 are not provided. When a person to be measured is placed on a body fat scale such that the soles of the feet of the person contact the current electrodes 50A and 50B and the voltage electrodes 51A and 51B so as to measure the percentage of body fat, assume that the diodes D11 and D22 for countermeasures against electrostatic breakdown break down or have already broken down due to static electricity. When VDD is +4 V and −VDD is −4 V, the diodes D11 and D22 are in a conduction state. Accordingly, a potential difference of 8 V is generated between the current electrode 50A and the current electrode 50B. Assuming that right and left tiptoes contact the current electrode 50A and the current electrode 50B, respectively, a voltage of 8 V is applied between the both feet of the human body. Assuming that the impedance between the both feet of the human body is about 500Ω, a direct current of 8 (V)/500 (Ω)=16 (mA) flows between the both feet of the human body. Although the person can get away from such a state, the effect of the current of 16 mA on the human body is considered to cause the person considerable distress.

Thus, in the case where a potential difference of several volts is generated between the electrodes when breakdown or failure occurs in an electrical component on the measurement circuit, if the human body contacts the electrodes at the time, a current of tens of mA may flow through the human body. This state occurs not only when the diodes for countermeasures against electrostatic breakdown break down, but also when static electricity is applied to the circuit, or when such a load that causes the body fat meter itself to be dropped is applied. In this case, a potential difference of several volts may be generated and the above-mentioned state may occur also when the constant current circuit operational amplifier U12 breaks down; the output of the operational amplifier becomes the voltage of VDD or −VDD; the analog switch IC1 breaks down; or the IC terminals internally connected to VDD or −VDD and each electrode plate comes into the conduction state, for example. Note that the impedance between the both feet varies depending on the person to be measured, and the rated voltages VDD and −VDD of the circuit also vary depending on the design method, measurement precision, and the like. Accordingly, the above-mentioned current does not always flow through every person, but there is also a possibility that a more current flows through a person to be measured.

Next, a case where the capacitors C1 to C4 are provided in the circuit will be described. To measure the percentage of body fat, when the person to be measured is placed on the body fat scale so that the soles of the feet contact the current electrodes 50A and 50B and the voltage electrodes 51A and 51B, assume that the diodes D11 and D22 for countermeasures against electrostatic breakdown break down or have already broken down due to static electricity. At this time, the diode D11 and the diode D22 are in the conduction state. However, even when the person to be measured is placed on the body fat scale so as to contact the current electrodes 50A and 50B, no direct current flows through the person because the capacitors C1 and C2 are disposed in the path. Accordingly, if the capacitors are disposed between each electrode and the measurement circuit, such an abnormality that a direct current of tens of mA flows through the human body can be avoided. During normal operation, an alternating current of 50 kHz and about 500 μA is used as a measurement current for the bioelectrical impedance. Accordingly, even when the capacitors are disposed in the measurement path, an alternating current can flow through the capacitors without causing any problem in the measurement. Further, when the frequency of the measurement current is 50 kHz and the capacity is equal to or greater than 0.1 μF, the capacitors C1 to C4 have no adverse influence on the measurement precision. As the type of the capacitors C1 to C4, an inexpensive ceramic capacitor commercially available may be used. Alternatively, an electrolytic capacitor, a film capacitor, or a chip component such as a pressure membrane chip resistor may also be used without causing any problem.

In addition, there is proposed a bioelectrical impedance detecting apparatus that detects an abnormality state in which a contact on each electrode of a living organism is incomplete or a contact resistance is large, thereby preventing a situation in which an erroneous impedance is calculated (Japanese Unexamined Patent Application Publication No. 2003-93361). Moreover, a biometric apparatus capable of reducing power consumption with a small and simple configuration (Japanese Unexamined Patent Application Publication No. 2009-82362).

### SUMMARY

However, the inventors of the present invention have found that the above-mentioned techniques have a problem. The body fat scale 300 disclosed in Japanese Unexamined Patent Application Publication No. 2001-161655 can prevent occurrence of an overcurrent, but requires a capacitor as an external component. This capacitor requires a relatively large capacitance value so as to avoid an adverse effect on the measurement precision. For example, when the frequency of the measurement current is 50 kHz and the alternating current has an amplitude of 500 μA, a capacitance value of 0.1 μF or more is generally required. It is difficult to incorporate such a capacitance value in a semiconductor device such as an LSI. Accordingly, it is necessary to use a ceramic capacitor, an electrolytic capacitor, or a film capacitor as an external component of the semiconductor device. This leads to an increase in cost of the bioelectrical impedance measuring apparatus.

A first aspect of the present invention is a bioelectrical impedance measuring apparatus including: a reference resistor; first and second current supply electrodes that contact a living organism to be measured; first and second voltage measurement electrodes that contact the living organism; a current supply unit that supplies a current between first and second current output terminals; a voltage measurement unit that measures a voltage input to first and second voltage input terminals; a first switch unit that connects the first current output terminal to one or both of the first current supply electrode and a first terminal of the reference resistor, and connects the second current output terminal to one or both of the second current supply electrode and a second terminal of the reference resistor; a second switch unit that connects the first voltage input terminal to one or both of the first voltage measurement electrode and the first terminal of the reference resistor, and connects the second voltage input terminal to one or both of the second voltage measurement electrode and the second terminal of the reference resistor; and a control circuit that controls the first and second switch units to control bioelectrical impedance measurement processing. The control circuit causes the first switch unit to connect the first current output terminal to the first terminal of the reference resistor, and to connect the second current output terminal to the second terminal of the reference resistor. The control circuit causes the second switch unit to connect the first voltage input terminal to the first terminal of the reference resistor, and to connect the second voltage input terminal to the second terminal of the reference resistor. The control circuit causes the voltage measurement unit to measure a voltage between the first voltage input terminal and the second voltage input terminal, to thereby perform self-diagnosis of a path through which a current flows. In the bioelectrical impedance measuring apparatus according to the first aspect of the present invention, the self-diagnosis is carried out prior to the measurement of a bioelectrical impedance, to thereby check the presence or absence of an abnormality in the path through which a current flows. This prevents an overcurrent from flowing through the living organism.

A second aspect of the present invention is a semiconductor device including: a reference resistor; a current supply unit that supplies a current between first and second current output terminals; a voltage measurement unit that measures a voltage input to first and second voltage input terminals; a first switch unit that connects the first current output terminal to one or both of the first current supply electrode and a first terminal of the reference resistor, and connects the second current output terminal to one or both of the second current supply electrode and a second terminal of the reference resistor, the first and second current supply electrodes being configured to contact a living organism to be measured; a second switch unit that connects the first voltage input terminal to one or both of the first voltage measurement electrode and the first terminal of the reference resistor, and connects the second voltage input terminal to one or both of the second voltage measurement electrode and the second terminal of reference resistor, the first and second voltage measurement electrodes being configured to contact the living organism; and a control circuit that controls the first and second switch units to control bioelectrical impedance measurement processing. The control circuit causes the first switch unit to connect the first current output terminal to the first terminal of the reference resistor, and to connect the second current output terminal to the second terminal of the reference resistor. The control circuit causes the second switch to connect the first voltage input terminal to the first terminal of the reference resistor, and to connect the second voltage input terminal to the second terminal of the reference resistor. The control circuit causes the voltage measurement unit to measure a voltage between the first voltage input terminal and the second voltage input terminal, to thereby perform self-diagnosis of a path through which a current flows. The semiconductor device according to the second aspect of the present invention carries out the self-diagnosis prior to the measurement of a bioelectrical impendence, to thereby check the presence or absence of an abnormality in the path through which a current flows. This prevents an overcurrent from flowing through the living organism.

A third aspect of the present invention is a control method for a bioelectrical impedance measuring apparatus, including: connecting a first current output terminal of a current supply unit to a first terminal of a reference resistor, the current supply unit being configured to supply a current between the first current output terminal and a second current output terminal; connecting the second current output terminal of the current supply unit to a second terminal of the reference resistor; connecting a first voltage input terminal of a voltage measurement unit to the first terminal of the reference resistor, the voltage measurement unit being configured to measure a voltage to be input to the first voltage input terminal and a second voltage input terminal; connecting the second voltage input terminal of the voltage measurement unit to the second terminal of the reference resistor; and measuring a voltage between the first voltage input terminal and the second voltage input terminal, to thereby perform self-diagnosis of a path through which a current flows. In the control method for a bioelectrical impedance measuring apparatus according to the third aspect of the present invention, the self-diagnosis is carried out prior to the measurement of the bioelectrical impedance to thereby check the presence or absence of an abnormality in the path through which a current flows. This prevents an overcurrent from flowing through a living organism.

According to the present invention, it is possible to provide a bioelectrical impedance measuring apparatus, a semiconductor device, and a control method for a bioelectrical impedance measuring apparatus, which are capable of performing self-diagnosis prior to measurement of a bioelectrical impedance, thereby preventing an overcurrent from flowing through a living organism.

### BRIEF DESCRIPTION OF THE DRAWINGS

The above and other aspects, advantages and features will be more apparent from the following description of certain embodiments taken in conjunction with the accompanying drawings, in which:

FIG. 1 is a circuit diagram schematically illustrating a configuration of a bioelectrical impedance measuring apparatus 100 according to a first embodiment;

FIG. 2 is a flowchart illustrating a measurement operation of the bioelectrical impedance measuring apparatus 100 according to the first embodiment;

FIG. 3A is an equivalent circuit diagram of the bioelectrical impedance measuring apparatus 100 in step S121;

FIG. 3B is an equivalent circuit diagram of the bioelectrical impedance measuring apparatus 100 in step S131;

FIG. 4A is a waveform chart of a voltage VR obtained when the bioelectrical impedance measuring apparatus 100 is in a normal state;

FIG. 4B is a waveform chart illustrating an example of the waveform of the voltage VR obtained when an abnormality occurs in the bioelectrical impedance measuring apparatus 100;

FIG. 4C is a waveform chart illustrating another example of the waveform of the voltage VR obtained when an abnormality occurs in the bioelectrical impedance measuring apparatus 100;

FIG. 5 is a flowchart illustrating a bioelectrical impedance measurement operation (step S23) in a measurement operation of a bioelectrical impedance measuring apparatus 200 according to a second embodiment;

FIG. 6 is an equivalent circuit diagram of the bioelectrical impedance measuring apparatus 200 in step S233; and

FIG. 7 is a block diagram illustrating an internal measurement circuit of a body fat scale 300 disclosed in Japanese Unexamined Patent Application Publication No. 2001-161655.

### DETAILED DESCRIPTION

Hereinafter, embodiments of the present invention will be described with reference to the drawings. In the drawings, the same elements are denoted by the same reference numerals, and a repeated explanation thereof is omitted as needed.

### First Embodiment

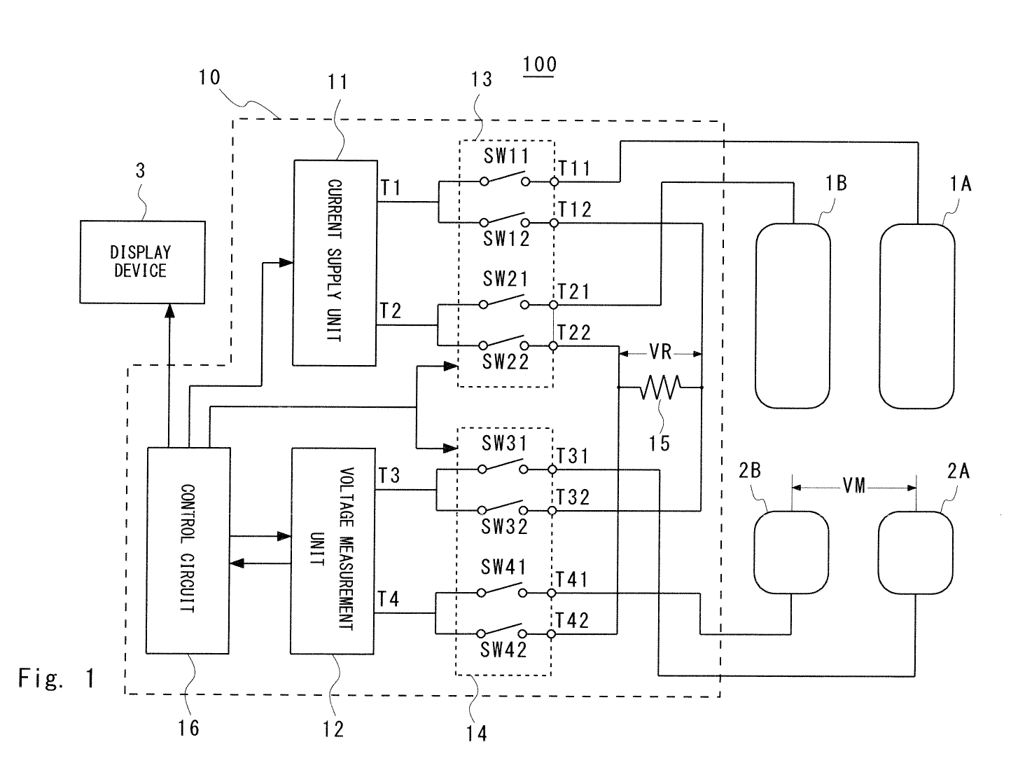

First, a bioelectrical impedance measuring apparatus 100 according to a first embodiment of the present invention will be described. FIG. 1 is a circuit diagram schematically illustrating the configuration of the bioelectrical impedance measuring apparatus 100 according to the first embodiment. As illustrated in FIG. 1, the bioelectrical impedance measuring apparatus 100 includes a semiconductor device 10, current supply electrodes 1A and 1B, voltage measurement electrodes 2A and 2B, and a display device 3.

The semiconductor device 10 includes a current supply unit 11, a voltage measurement unit 12, switch units 13 and 14, a reference resistor 15, and a control circuit 16. The current supply unit 11 receives a command from the control circuit 16 and supplies a current through the switch unit 13. The voltage measurement unit 12 measures a voltage input through the switch unit 14, and outputs the measurement result to the control circuit 16.

The switch unit 13 includes switches SW11, SW12, SW21, and SW22. The switch SW11 is disposed between a terminal T11 and a terminal T1 of the current supply unit 11. The terminal T11 is connected to the current supply electrode 1A. The switch SW12 is disposed between a terminal T12 and the terminal T1 of the current supply unit 11. The switch SW21 is disposed between a terminal T21 and a terminal T2 of the current supply unit 11. The terminal T21 is connected to the current supply electrode 1B. The switch SW22 is disposed between a terminal T22 and the terminal T2 of the current supply unit 11. The operation of the switch unit 13, i.e., the opening and closing of the switches SW11, SW12, SW21, and SW22, is controlled by the control circuit 16.

The switch unit 14 includes switches SW31, SW32, SW41, and SW42. The switch SW31 is disposed between a terminal T31 and a terminal T3 of the voltage measurement unit 12. The terminal 131 is connected to the voltage measurement electrode 2A. The switch SW32 is disposed between a terminal T32 and the terminal T3 of the voltage measurement unit 12. The switch SW41 is disposed between a terminal T41 and a terminal T4 of the voltage measurement unit 12. The terminal T41 is connected to the voltage measurement electrode 2B. The switch SW42 is disposed between a terminal T42 and the terminal T4 of the voltage measurement unit 12. The operation of the switch unit 14, i.e., the opening and closing of the switches SW31, SW32, SW41, and SW42, is controlled by the control circuit 16.

The terminal T12 of the switch unit 13 is connected to the terminal T32 of the switch unit 14. The terminal T22 of the switch unit 13 is connected to the terminal T42 of the switch unit 14. The reference resistor 15 is connected between one node which is connected to the terminal T12 of the switch unit 13 and the terminal T32 of the switch unit 14 and the other node which is connected to the terminal T22 of the switch unit 13 and the terminal T42 of the switch unit 14. The reference resistor 15 has a resistance value of 1 kΩ, for example.

The current supply electrodes 1A and 1B and the voltage measurement electrodes 2A and 2B are electrodes that contact a living organism to be measured. When the living organism is a human body, for example, the sole of the right foot contacts the current supply electrode 1A and the voltage measurement electrode 2A, and the sole of the left foot contacts the current supply electrode 1B and the voltage measurement electrode 2B. This allows a current to flow through the living organism through the current supply electrodes 1A and 1B to thereby measure the voltage of the living organism between the voltage measurement electrodes 2A and 2B.

As described above, the control circuit 16 controls the operations of the current supply unit 11 and the switch units 13 and 14. The control circuit 16 causes the display device 3 to display a measured value or a warning according to the measurement result of the voltage measurement unit 12.

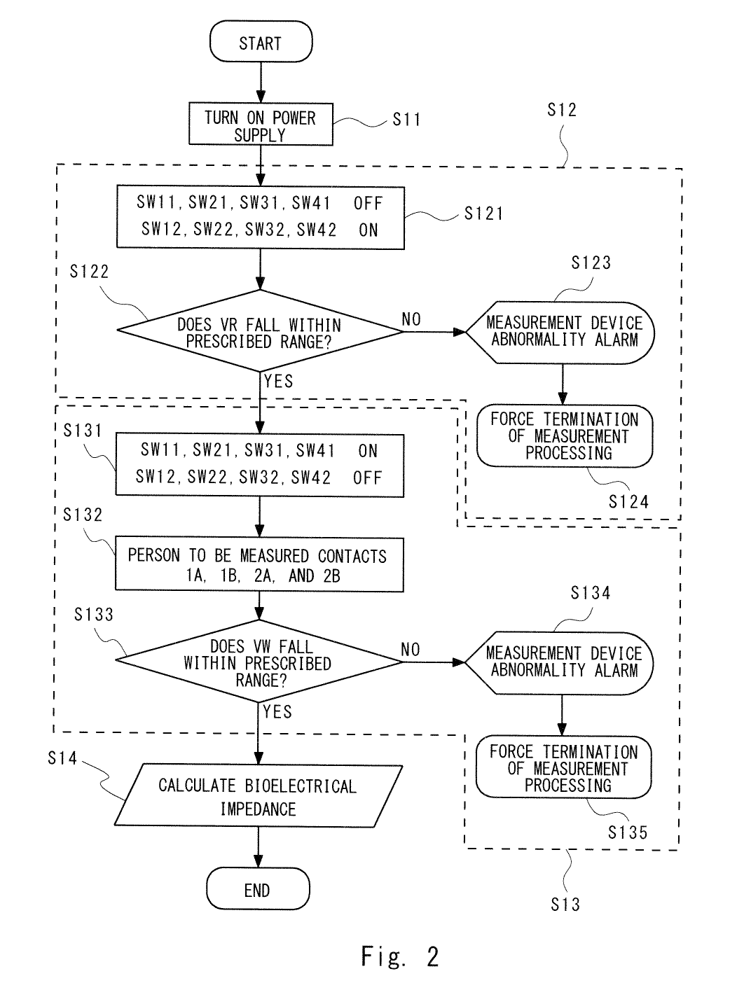

Subsequently, a measurement operation of the bioelectrical impedance measuring apparatus 100 will be described. FIG. 2 is a flowchart illustrating the measurement operation of the bioelectrical impedance measuring apparatus 100 according to the first embodiment. The bioelectrical impedance measuring apparatus 100 is initially in a power-off state, and all the switches SW11, SW12, SW21, SW22, SW31, SW32, SW41, and SW42 are turned off. In the measurement operation of the bioelectrical impedance measuring apparatus 100, after the power supply is turned on (step S11), a self-diagnosis operation (step S12), a bioelectrical impedance measurement operation (step S13), and a bioelectrical impedance calculation operation (step S14) are carried out. The steps will be described in detail below.

(Step S11)

To start the measurement, the power supply of the bioelectrical impedance measuring apparatus 100 is turned on. In this case, a person to be measured, whose fat mass is to be measured, pushes down a power supply switch, for example, thereby turning on the power supply of the bioelectrical impedance measuring apparatus 100. When the power supply is turned on, the current supply unit 11 comes into an alternating current supply state and the voltage measurement unit 12 comes into a voltage detection state. Note that the method for turning on the power supply is not limited to this example.

(Step S12)

The self-diagnosis operation includes steps S121 to S124.

(Step S121)

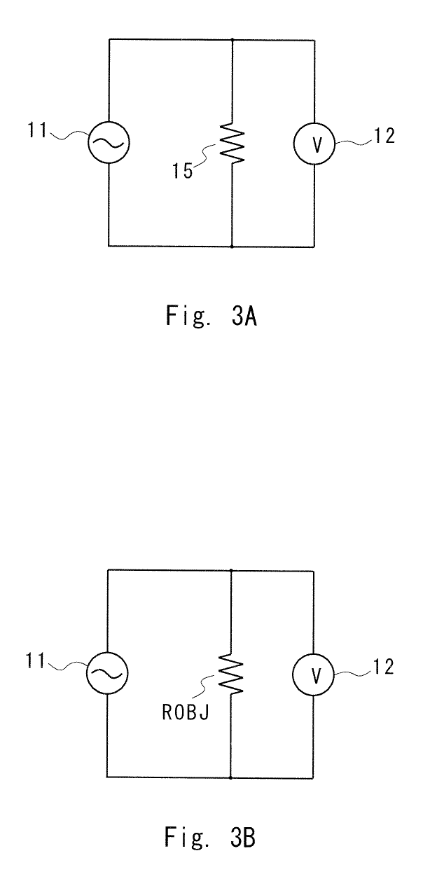

The control circuit 16 turns on the switches SW12, SW22, SW32, and SW42. FIG. 3A is an equivalent circuit diagram of the bioelectrical impedance measuring apparatus 100 in step S121. In step S121, a current flows through a path (hereinafter, referred to as a current supply path) represented as the terminal T1 of the current supply unit 11→the switch SW12→the terminal T12→the reference resistor 15→the terminal T22→the switch SW22→the terminal T2 of the current supply unit 11. The terminal T3 of the voltage measurement unit 12 is connected to the terminal 112 through the switch SW32 and the terminal T32. The terminal T4 of the voltage measurement unit 12 is connected to the terminal T22 through the switch SW42 and the terminal T42. Accordingly, the voltage measurement unit 12 measures a voltage VR between both ends of the reference resistor 15, and outputs the measurement result to the control circuit 16.

(Step S122)

The control circuit 16 measures the voltage VR to check whether the voltage VR falls within a prescribed range. When the voltage VR falls within the prescribed range, the process proceeds to the bioelectrical impedance measurement operation (step S13). When the voltage VR falls outside the prescribed range, the process proceeds to step S123.

(Step S123)

When the voltage VR falls outside the prescribed range, the control circuit 16 determines that an abnormality is occurring in the current supply path, and outputs a measurement device abnormality alarm. This measurement device abnormality alarm is output to the display device 3, for example. The display device 3 displays the measurement device abnormality alarm on a display screen, for example, thereby allowing the person to be measured to recognize that an abnormality is occurring in the current supply path of the bioelectrical impedance measuring apparatus 100. In other words, the measurement device abnormality alarm warns the person to be measured not to contact the current supply electrodes A and 1B and the voltage measurement electrodes 2A and 2B. This prevents an excessive current, which is generated due to abnormality in the current supply path, from flowing through the person to he measured.

(Step S124)

After outputting the measurement device abnormality alarm, the control circuit 16 forcefully terminates the measurement processing. For example, the control circuit 16 turns off the switches SW12, SW22, SW32, and SW42, thereby interrupting the current flowing through the current supply path. Further, for example, the control circuit 16 commands the current supply unit 11 to stop supplying the current. Specifically, the control circuit 16 interrupts the current flowing through the current supply path, thereby preventing the situation in which an excessive current, which is generated due to an abnormality in the current supply path, from flowing through the person to be measured.

(Step S13)

The impedance measurement operation (step S13) includes steps S131 to S135.

(Step S131)

When the voltage VR falls within the prescribed range, the control circuit 16 turns off the switches SW12, SW22, SW32, and SW42 and turns on the switches SW11, SW21, SW31, and SW41. FIG. 3B is an equivalent circuit diagram of the bioelectrical impedance measuring apparatus 100 in step S131. In step S131, the terminal T1 of the current supply unit 11 is connected to the current supply electrode 1A through the switch SW11 and the terminal T11. The terminal T2 of the current supply unit 11 is connected to the current supply electrode 1B through the switch SW21 and the terminal T21. The terminal T3 of the voltage measurement unit 12 is connected to the voltage measurement electrode 2A through the switch SW31 and the terminal T31. The terminal T4 of the voltage measurement unit 12 is connected to the voltage measurement electrode 2B through the switch SW41 and the terminal T41. Thus, preparation for the measurement is completed.

(Step S132)

The control circuit 16 notifies the display device 3 of the completion of preparation for the measurement. The display device 3 displays information indicating the completion of preparation for the measurement on the display screen, for example. Accordingly, to measure the bioelectrical impedance, the person to be measured contacts the current supply electrodes 1A and 1B and the voltage measurement electrodes 2A and 2B.

(Step S133)

When the person to be measured contacts the current supply electrodes 1A and 1B and the voltage measurement electrodes 2A and 2B, a measurement current Im flows through a path represented as the terminal T1 of the current supply unit 11→the switch SW11→the terminal T11→the current supply electrode 1A→the person to be measured (indicated by a resistor ROBJ in FIG. 3B)→the current supply electrode 1B the terminal T21→the switch SW21→the terminal T2 of the current supply unit 11. As a result, the measurement current Im flows through the person to be measured. The voltage measurement unit 12 measures a voltage VM between both ends of the resistor ROBJ, and outputs the measurement result to the control circuit 16. The control circuit 16 determines whether the voltage VM falls within the prescribed range. When the voltage VM falls outside the prescribed range, the process proceeds to step S134. When the voltage VM falls within the prescribed range, the process proceeds to the bioelectrical impedance calculation operation (step S114).

(Step S134)

When the voltage VM falls outside the prescribed range, the control circuit 16 determines that an abnormality is occurring in the bioelectrical impedance measuring apparatus 100, and outputs the measurement device abnormality alarm. As described above, the reference resistor 15 has a resistance value of 1 kΩ. When the person to be measured is a human, the value of the bioelectrical impedance of the human is typically about 1 kΩ. Accordingly, when the voltage VM is equal to the voltage VR measured in step S122, it can be determined that the measured value of the voltage VM is normal.

A specific example of the voltage VM will now be described. Assume herein that the reference resistor 15 has a resistance value of 1 kΩ; the current supplied from the current supply unit 11 has a frequency of 50 kHz; and the current has an amplitude of 500 μA. At this time, the voltage VR is given by the following expression (1).

$$\begin{matrix}VR = 1000 \{ Ω ] \times 500 [ μ A ] \times sin ( t ) \\ = 0.5 [ V ] sin ( t )\end{matrix} \qquad ( 1 )$$

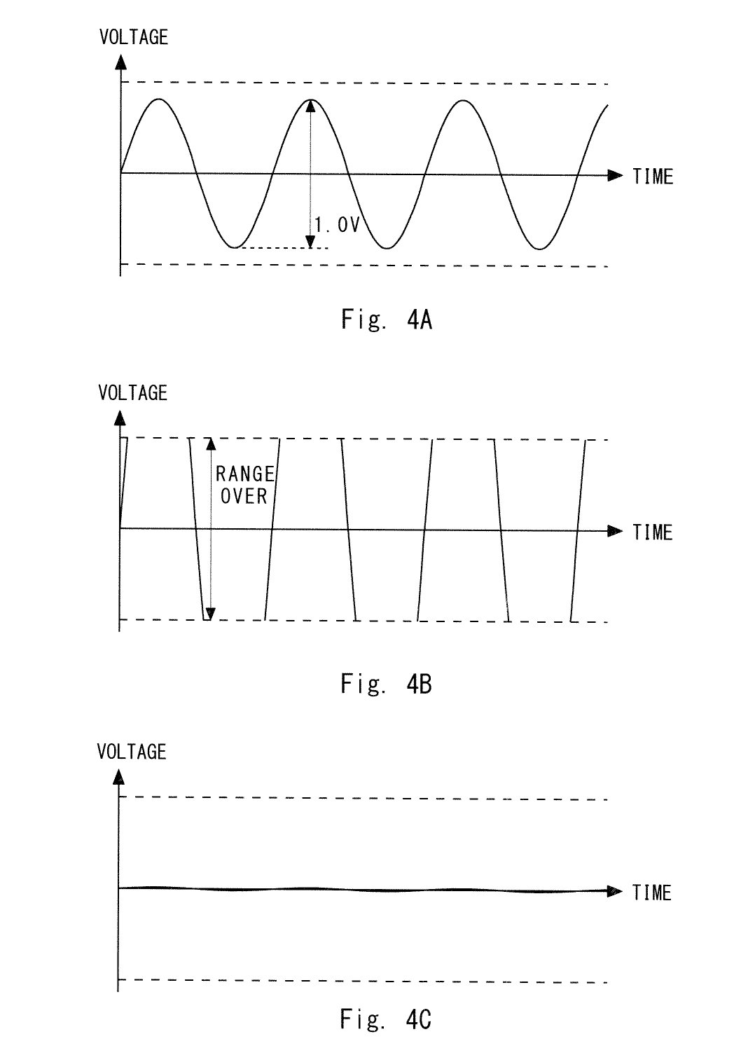

FIG. 4A is a waveform chart of the voltage VR obtained when the bioelectrical impedance measuring apparatus 100 is in a normal state. As illustrated in FIG. 4A, when the bioelectrical impedance measuring apparatus 100 is in the normal state, the voltage VR shows a sine waveform of a voltage amplitude (0.5 V) given by Expression (1).

FIG. 4B is a waveform chart illustrating an example of the waveform of the voltage VR obtained when an abnormality occurs in the bioelectrical impedance measuring apparatus 100. As illustrated in FIG. 4B, for example, when the current supplied from the current supply unit 11 is excessively large (overcurrent), or when the reference resistor 15 is open, the value of the voltage PR is excessively large, which causes an over-range state.

FIG. 4C is a waveform chart illustrating another example of the waveform of the voltage VR obtained when an abnormality occurs in the bioelectrical impedance measuring apparatus 100. As illustrated in FIG. 4C, for example, when excessively small or no current is supplied from the current supply unit 11, or when the reference resistor 15 is short-circuited, the voltage VR has a small value.

As described above, the amplitude of the voltage VR in step S134 is observed, thereby enabling detection of an abnormality in the bioelectrical impedance measuring apparatus 100.

Note that since there is an individual difference between persons to be measured, the normal value of the voltage VM has a certain range. In other words, a distribution range of values of the bioelectrical impedance obtained in consideration of the individual difference between persons to be measured may be set as the prescribed range of the voltage VM. The measurement device abnormality alarm is output to the display device 3, for example, thereby allowing the person to be measured to recognize that an abnormality is occurring in the bioelectrical impedance measuring apparatus 100 and the normal bioelectrical impedance measurement cannot be carried out.

(Step S135)

After outputting the measurement device abnormality alarm, the control circuit 16 forcefully terminates the measurement processing. The method for forcefully terminating the measurement processing is similar to step S124, so the description is omitted.

(Step S14)

When the voltage VM falls within the prescribed range, the control circuit 16 calculates the bioelectrical impedance from the values of the current Im and the voltage VM. The control circuit 16 calculates the fat mass (percentage of body fat) of the person to be measured based on the living organism impedance and feature values of the person to be measured (e.g., age, height, gender, and weight, in the case where the person to be measured is a human), and outputs the calculated value as the measurement result. The measurement result is output to the display device 3, for example. The display device 3 displays, as the measurement result, the fat mass (percentage of body fat) of the person to be measured, on the display screen, for example. This allows the person to be measured to recognize the fat mass (percentage of body fat) of the person. Thus, the measurement operation of the bioelectrical impedance measuring apparatus 100 is completed.

According to the configuration and method of the present invention as described above, a failure in the current supply path is found by self-diagnosis, thereby preventing an excessive current from flowing through the person to be measured.

Accordingly, it is possible to provide a bioelectrical impedance measuring apparatus that performs self-diagnosis prior to a bioelectrical impedance measurement to detect an abnormality in a path through which a current flows, thereby preventing an overcurrent from flowing through a person to be measured, a semiconductor device mounted on the bioelectrical impedance measuring apparatus, and a control method for the bioelectrical impedance measuring apparatus.

This configuration requires no external components such as a capacitor. Accordingly, the bioelectrical impedance measuring apparatus can be achieved at low cost.

### Second Embodiment

Next, a bioelectrical impedance measuring apparatus 200 according to a second embodiment of the present invention will be described. The bioelectrical impedance measuring apparatus 200 has a configuration similar to that of the bioelectrical impedance measuring apparatus 100 according to the first embodiment. However, the bioelectrical impedance measurement operation of the bioelectrical impedance measuring apparatus 200 differs from that of the bioelectrical impedance measuring apparatus 100.

The measurement operation of the bioelectrical impedance measuring apparatus 200 will be described. The bioelectrical impedance measuring apparatus 200 is initially in the power-off state, and all the switches SW11, SW12, SW21, SW22, SW31, SW32, SW41, and SW42 are turned off. In the measurement operation of the bioelectrical impedance measuring apparatus 200, after the power supply is turned on (step S21), a self-diagnosis operation (step S22), a bioelectrical impedance measurement operation (step S23), and a bioelectrical impedance calculation operation (step S24) are carried out. Note that the operations in steps S21, S22, and S24 are respectively similar to steps S11, S12, and S14 illustrated in FIG. 2, so the description thereof is omitted.

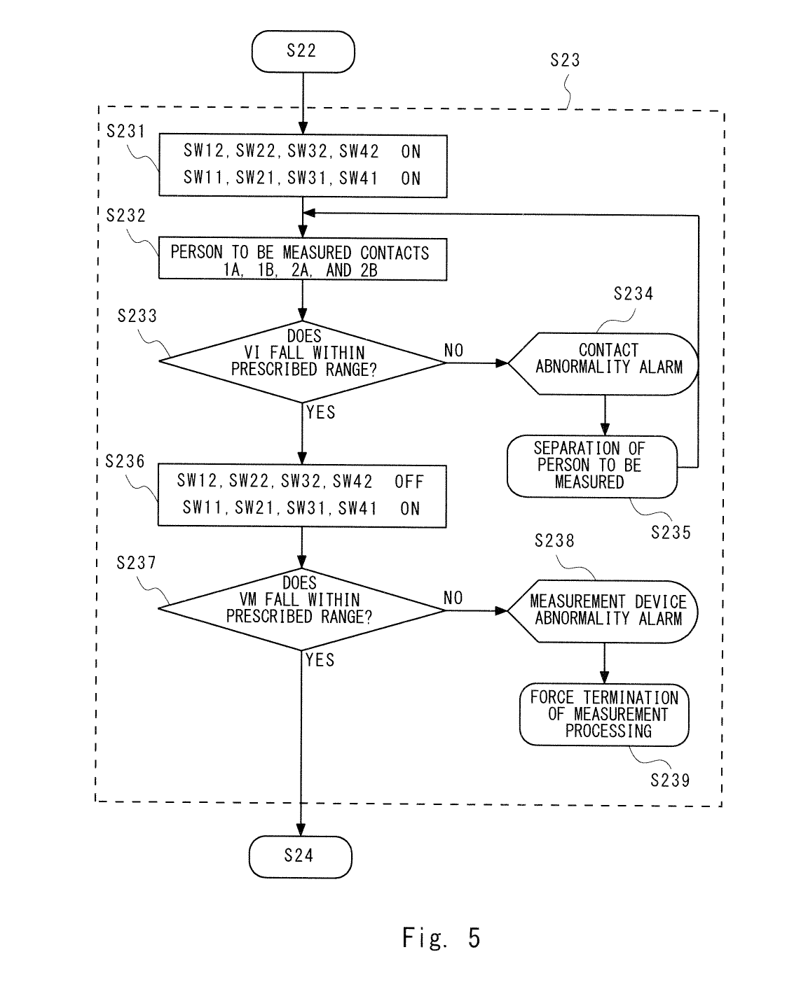

The bioelectrical impedance measurement operation (step S23) of the bioelectrical impedance measuring apparatus 200 will be described below. FIG. 5 is a flowchart illustrating the bioelectrical impedance measurement operation (step S23) in the measurement operation of the bioelectrical impedance measuring apparatus 200 according to the second embodiment.

(Step S23)

The impedance measurement operation (step S23) in the measurement operation of the bioelectrical impedance measuring apparatus 200 includes steps S231 to S239.

(Step S231)

When the voltage VR falls within the prescribed range, the control circuit 16 turns on the switches SW11, SW12, SW21, SW22, SW31, SW32, SW41, and SW42. In step S231, the terminal T1 of the current supply unit 11 is connected to the current supply electrode 1A through the switch SW11 and the terminal T11. The terminal T1 of the current supply unit 11 is also connected to the terminal T3 of the voltage measurement unit 12 through the switch SW12, the terminal T12, the terminal T32, and the switch SW32. The terminal T3 of the voltage measurement unit 12 is connected to the voltage measurement electrode 2A through the switch SW31 and the terminal T31.

The terminal T2 of the current supply unit 11 is connected to the current supply electrode 1B through the switch SW21 and the terminal T21. The terminal T2 of the current supply unit 11 is also connected to the terminal T4 of the voltage measurement unit 12 through the switch SW22, the terminal T22, the terminal T42, and the switch SW42. The terminal T4 of the voltage measurement unit 12 is connected to the voltage measurement electrode 2B through the switch SW41 and the terminal T41.

In this case, the path between the current supply electrode 1A and the current supply electrode 1B is open, and the path between the voltage measurement electrode 2A and the voltage measurement electrode 2B is also open. Accordingly, the equivalent circuit diagram in step S231 is similar to FIG. 3A. In this case, a current flows through a path represented as the terminal T1 of the current supply unit 11→the switch SW12→the terminal T12→the reference resistor 15→the terminal T22→the switch SW22→the terminal T2 of the current supply unit 11. This allows the voltage measurement unit 12 to detect the voltage VR, which is measured in step S22, between both ends of the reference resistor 15. Thus, preparation for the measurement is completed.

(Step S232)

The control circuit 16 notifies the display device 3 of the completion of preparation for the measurement. The display device 3 displays information indicating the completion of preparation for the measurement on the display screen, for example. Thus, to measure the bioelectrical impedance, the person to be measured contacts the current supply electrodes 1A and 1B and the voltage measurement electrodes 2A and 2B.

(Step S233)

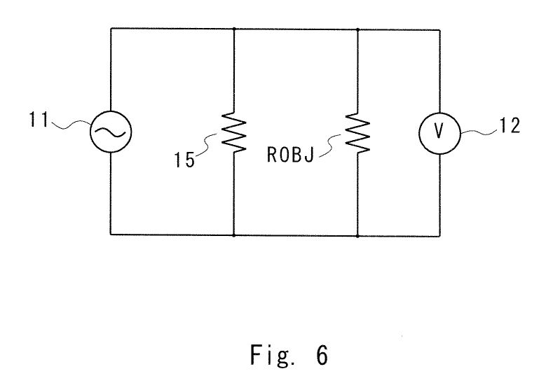

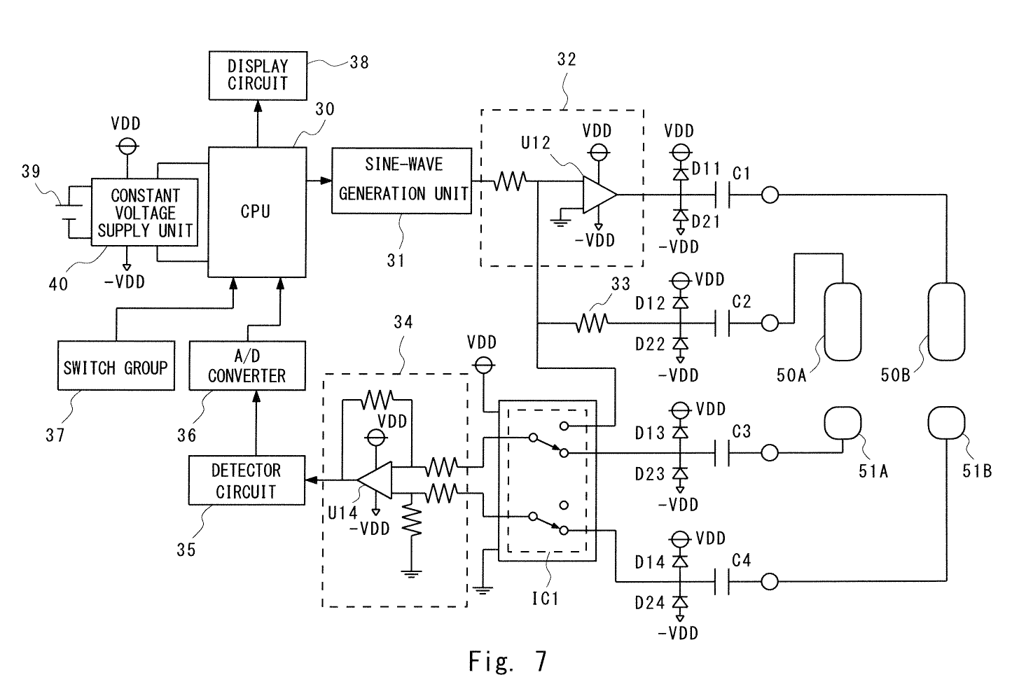

When the person to be measured contacts the current supply electrodes 1A and 1B and the voltage measurement electrodes 2A and 2B, a current also flows through a path represented as the terminal T1 of the current supply unit 11→the switch SW11→the terminal T11→the current supply electrode 1A→the person to be measured (indicated by the resistor ROBJ in FIG. 3B)→the current supply electrode 1B→the terminal T21→the switch SW21the the terminal 12 of the current supply unit 11. As a result, the measurement current Im flows through the person to be measured. FIG. 6 is an equivalent circuit diagram of the bioelectrical impedance measuring apparatus 200 in step S233. The voltage measurement unit 12 measures a voltage VC between both ends of a combined resistor including the resistor ROBJ and the reference resistor 15, which are connected in parallel, and outputs the measurement result to the control circuit 16. The control circuit 16 determines whether the voltage VC falls within the prescribed range. When the voltage VC falls outside the prescribed range, the process proceeds to step S234. When the voltage VC falls within the prescribed range, the process proceeds to step S236.

(Step S234)

When the voltage VC falls outside the prescribed range, the control circuit 16 determines that the contact state between the person to be measured and the current supply electrodes 1A and 1B and the voltage measurement electrodes 2A and 2B is unfavorable, and outputs a contact abnormality alarm. As described above, the reference resistor 15 has a resistance value of 1 kΩ. When the person to be measured is a human, the value of the bioelectrical impedance of the human is typically about 1 kΩ. Accordingly, the combined resistor including the resistor ROBJ and the reference resistor 15, which are connected in parallel, has a resistance value of 500Ω. In other words, if the contact state between the person to be measured and the current supply electrodes 1A and 1B and the voltage measurement electrodes 2A and 2B is favorable, the voltage VC is about a half of the voltage VR. That is, when the value of the voltage VC is not a half of the voltage VR, it can be determined that contact abnormality is occurring. Note that since there is an individual difference between persons to be measured, the normal value of the voltage VC has a certain range. In other words, a distribution range of values of the bioelectrical impedance obtained in consideration of the individual difference between persons to be measured may be set as the prescribed range of the voltage VC. The contact abnormality alarm is output to the display device 3, for example.

(Step S235)

The display device 3 displays the contact abnormality alarm on the display screen, for example, thereby allowing the person to be measured to recognize the contact abnormality and start over the contact operation. For example, the person to be measured temporarily moves away from the current supply electrodes 1A and 1B and the voltage measurement electrodes 2A and 2B.

Then, returning to step S232, the person to be measured can retry contacting the current supply electrodes 1A and 1B and the voltage measurement electrodes 2A and 2B. Specifically, steps S232 to S235 involve loop processing, and this loop processing reliably achieves a favorable contact state prior to the measurement of the bioelectrical impedance.

(Step S236)

When the voltage VC falls within the prescribed range, the control circuit 16 turns off the switches SW12, SW22, SW32, and SW42 and turns on the switches SW11, SW21, SW31, and SW41. Accordingly, the equivalent circuit diagram in step S236 is similar to FIG. 3B. That is, the operation in step S236 is similar to that in step S131 illustrated in FIG. 2, so the description is omitted.

(Steps S237 to S239)

Steps S237 to S239 are respectively similar to steps S133 to S135 illustrated in FIG. 2, so the description thereof is omitted.

According to the configuration and method of the present invention as described above, a failure in the current supply path is found by self-diagnosis, thereby preventing an excessive current from flowing through the person to be measured. Further, according to the configuration and method of the present invention, the contact between the person to be measured and the current supply electrodes and the voltage measurement electrodes can be maintained in a favorable state, and the bioelectrical impedance can be measured more accurately.

Note that the present invention is not limited to the above embodiments, but can be modified as needed without departing from the scope of the invention. For example, the configurations of the switch units 13 and 14 described in the embodiments described above are 13 illustrated by way of example only. Accordingly, the switch units 13 and 14 may have other configurations, as long as the switch units can carry out similar operations.

The first and second embodiments can be combined as desirable by one of ordinary skill in the art.

While the invention has been described in terms of several embodiments, those skilled in the art will recognize that the invention can be practiced with various modifications within the spirit and scope of the appended claims and the invention is not limited to the examples described above.

Further, the scope of the claims is not limited by the embodiments described above.

Furthermore, it is noted that, Applicant's intent is to encompass equivalents of all claim elements, even if amended later during prosecution.

What is claimed is:

1. A bioelectrical impedance measuring apparatus comprising:
a reference resistor;
first and second current supply electrodes that contact a living organism to be measured;
first and second voltage measurement electrodes that contact the living organism;
a current supply unit that supplies a current between first and second current output terminals;
a voltage measurement unit that measures a voltage input to first and second voltage input terminals;
a first switch unit that connects the first current output terminal to one or both of the first current supply electrode and a first terminal of the reference resistor, and connects the second current output terminal to one or both of the second current supply electrode and a second terminal of the reference resistor;
a second switch unit that connects the first voltage input terminal to one or both of the first voltage measurement electrode and the first terminal of the reference resistor, and connects the second voltage input terminal to one or both of the second voltage measurement electrode and the second terminal of the reference resistor; and
a control circuit that controls the first and second switch units to control bioelectrical impedance measurement processing, wherein
the control circuit causes the first switch unit to connect the first current output terminal to the first terminal of the reference resistor, and to connect the second current output terminal to the second terminal of the reference resistor,
the control circuit causes the second switch unit to connect the first voltage input terminal to the first terminal of the reference resistor, and to connect the second voltage input terminal to the second terminal of the reference resistor, and
the control circuit causes the voltage measurement unit to measure a voltage between the first voltage input terminal and the second voltage input terminal, to thereby perform self-diagnosis of a path through which a current flows.

2. The bioelectrical impedance measuring apparatus according to claim 1, wherein the control circuit detects an abnormality in the path through which a current flows, when the voltage between the first voltage input terminal and the second voltage input terminal falls outside a predetermined range.

3. The bioelectrical impedance measuring apparatus according to claim 2, wherein the control circuit terminates the bioelectrical impedance measurement processing, upon detection of an abnormality in the path through which a current flows.

4. The bioelectrical impedance measuring apparatus according to claim 1, wherein after completion of the self-diagnosis, the control circuit causes the first switch unit to connect the first current output terminal to the first current supply electrode, and to connect the second current output terminal to the second current supply electrode, causes the second switch unit to connect the first voltage input terminal to the first voltage measurement electrode, and to connect the second voltage input terminal to the second voltage measurement electrode, and causes the voltage measurement unit to measure a voltage between the first voltage input terminal and the second voltage input terminal, to thereby measure an impedance of the living organism.

5. The bioelectrical impedance measuring apparatus according to claim 4, wherein prior to the measurement of the impedance of the living organism, the control circuit causes the first switch unit to connect the first current output terminal to the first current supply electrode and the first terminal of the reference resistor, and to connect the second current output terminal to the second current supply electrode and the second terminal of the reference resistor, causes the second switch unit to connect the first voltage input terminal to the first voltage measurement electrode and the first terminal of the reference resistor, and to connect the second voltage input terminal to the second voltage measurement electrode and the second terminal of the reference resistor, and causes the voltage measurement unit to measure a voltage between the first voltage input terminal and the second voltage input terminal, to thereby diagnose a contact state between the living organism and the first and second current supply electrodes and the first and second voltage measurement electrodes.

6. The bioelectrical impedance measuring apparatus according to claim 5, wherein the control circuit detects a fluctuation in the voltage between the first voltage input terminal and the second voltage input terminal, and detects an abnormality in the contact state between the living organism and the first and second current supply electrodes and the first and second voltage measurement electrodes when the voltage obtained after the fluctuation falls outside a predetermined range.

7. The bioelectrical impedance measuring apparatus according to claim 6, wherein the control circuit outputs an alarm upon detection of an abnormality in the contact state between the living organism and the first and second current supply electrodes and the first and second voltage measurement electrodes.

8. A semiconductor device comprising:
a reference resistor;
a current supply unit that supplies a current between first and second current output terminals;
a voltage measurement unit that measures a voltage input to first and second voltage input terminals;
a first switch unit that connects the first current output terminal to one or both of the first current supply electrode and a first terminal of the reference resistor, and connects the second current output terminal to one or both of the second current supply electrode and a second terminal of the reference resistor, the first and second current supply electrodes being configured to contact a living organism to be measured;
a second switch unit that connects the first voltage input terminal to one or both of the first voltage measurement electrode and the first terminal of the reference resistor, and connects the second voltage input terminal to one or both of the second voltage measurement electrode and the second terminal of the reference resistor, the first and second voltage measurement electrodes being configured to contact the living organism; and
a control circuit that controls the first and second switch units to control bioelectrical impedance measurement processing, wherein
the control circuit causes the first switch unit to connect the first current output terminal to the first terminal of the reference resistor, and to connect the second current output terminal to the second terminal of the reference resistor,
the control circuit causes the second switch to connect the first voltage input terminal to the first terminal of the reference resistor, and to connect the second voltage input terminal to the second terminal of the reference resistor, and
the control circuit causes the voltage measurement unit to measure a voltage between the first voltage input terminal and the second voltage input terminal, to thereby perform self-diagnosis of a path through which a current flows.

9. The semiconductor device according to claim 8, wherein the control circuit detects an abnormality in the path through which a current flows, when the voltage between the first voltage input terminal and the second voltage input terminal falls outside a predetermined range.

10. The semiconductor device according to claim 9, wherein the control circuit terminates the bioelectrical impedance measurement processing, upon detection of an abnormality in the path through which a current flows.

11. The semiconductor device according to claim 8, wherein after completion of the self-diagnosis, the control circuit causes the first switch unit to connect the first current output terminal to the first current supply electrode, and to connect the second current output terminal to the second current supply electrode, causes the second switch unit to connect the first voltage input terminal to the first voltage measurement electrode, and to connect the second voltage input terminal to the second voltage measurement electrode, and causes the voltage measurement unit to measure a voltage between the first voltage input terminal and the second voltage input terminal, to thereby measure an impedance of the living organism.

12. The semiconductor device according to claim 11, wherein prior to the measurement of the impedance of the living organism, the control circuit causes the first switch unit to connect the first current output terminal to the first current supply electrode and the first terminal of the reference resistor, and to connect the second current output terminal to the second current supply electrode and the second terminal of the reference resistor, causes the second switch unit to connect the first voltage input terminal to the first voltage measurement electrode and the first terminal of the reference resistor, and to connect the second voltage input terminal to the second voltage measurement electrode and the second terminal of the reference resistor, and causes the voltage measurement unit to measure a voltage between the first voltage input terminal and the second voltage input terminal, to thereby diagnose a contact state between the living organism and the first and second current supply electrodes and the first and second voltage measurement electrodes.

13. The semiconductor device according to claim 12, wherein the control circuit detects a fluctuation in the voltage between the first voltage input terminal and the second voltage input terminal, and detects an abnormality in the contact state between the living organism and the first and second current supply electrodes and the first and second voltage measurement electrodes when the voltage obtained after the fluctuation falls outside a predetermined range.

14. The semiconductor device according to claim 13, wherein the control circuit outputs an alarm upon detection of an abnormality in the contact state between the living organism and the first and second current supply electrodes and the first and second voltage measurement electrodes.

15. A control method for a bioelectrical impedance measuring apparatus, comprising:
connecting a first current output terminal of a current supply unit to a first terminal of a reference resistor, the current supply unit being configured to supply a current between the first current output terminal and a second current output terminal;
connecting the second current output terminal of the current supply unit to a second terminal of the reference resistor;
connecting a first voltage input terminal of a voltage measurement unit to the first terminal of the reference resistor, the voltage measurement unit being configured to measure a voltage to be input to the first voltage input terminal and a second voltage input terminal;
connecting the second voltage input terminal of the voltage measurement unit to the second terminal of the reference resistor; and
measuring a voltage between the first voltage input terminal and the second voltage input terminal, to thereby perform self-diagnosis of a path through which a current flows.

16. The control method for a bioelectrical impedance measuring apparatus according to claim 15, further comprising detecting a case where the voltage between the first voltage input terminal and the second voltage input terminal falls outside a predetermined range, as an abnormality in the path through which a current flows.

17. The control method for a bioelectrical impedance measuring apparatus according to claim 16, further comprising terminating the bioelectrical impedance measurement processing upon detection of an abnormality in the path through which a current flows.

18. The control method for a bioelectrical impedance measuring apparatus according to claim 15, further comprising, after completion of the self-diagnosis:
connecting the first current output terminal to a first current supply electrode that contacts a living organism to be measured;
connecting the second current output terminal to a second current supply electrode that contacts the living organism;
connecting the first voltage input terminal to a first voltage measurement electrode that contacts the living organism;
connecting the second voltage input terminal to a second voltage measurement electrode that contacts the living organism; and
measuring a voltage between the first voltage input terminal and the second voltage input terminal, to thereby measure an impedance of the living organism.

19. The control method for a bioelectrical impedance measuring g apparatus according to claim 18, further comprising, prior to the measurement of the impedance of the living organism:
connecting the first current output terminal to the first current supply electrode and the first terminal of the reference resistor;
connecting the second current output terminal to the second current supply electrode and the second terminal of the reference resistor;
connecting the first voltage input terminal to the first voltage measurement electrode and the first terminal of the reference resistor;
connecting the second voltage input terminal to the second voltage measurement electrode and the second terminal of the reference resistor; and
measuring a voltage between the first voltage input terminal and the second voltage input terminal, to thereby diagnose a contact state between the living organism and the first and second current supply electrodes and the first and second voltage measurement electrodes.

20. The control method for a bioelectrical impedance measuring apparatus according to claim 19, further comprising:
detecting a fluctuation in the voltage between the first voltage input terminal and the second voltage input terminal; and
detecting a case where the voltage obtained after the fluctuation falls outside a predetermined range, as an abnormality in the contact state between the living organism and the first and second current supply electrodes and the first and second voltage measurement electrodes.

21. The control method for a bioelectrical impedance measuring apparatus according to claim 20, further comprising outputting an alarm upon detection of an abnormality in the contact state between the living organism and the first and second current supply electrodes and the first and second voltage measurement electrodes.
